(19) United States
(12) Patent Application Publication
(10) Pub. No.: US 2010/0204093 A1
(43) Pub. Date: Aug. 12, 2010

(54) USE OF HEAT SHOCK ACTIVATORS FOR TISSUE REGENERATION

(75) Inventors: Shalesh Kaushal, Gainsville, FL (US); Maria G. Grant, Archer, FL (US)

Correspondence Address: EDWARDS ANGELL PALMER & DODGE LLP, P.O. BOX 55874, BOSTON, MA, 02205, US

(73) Assignee: University of Florida Research Foundation, Inc, Gainsville, FL (US)

(21) Appl. No.: 12/375,368

(22) PCT Filed: Jul. 27, 2007
(86) PCT No.: PCT/US2007/016991

§ 371 Date: Mar. 16, 2010

Related U.S. Application Data
(60) Provisional application No. 60/833,898, filed on Jul. 27, 2006.

(51) Int. Cl. A61K 38/19; A61K 31/395; A61K 31/335; A61K 31/121; A61K 31/7088; A61P 9/10

(52) U.S. Cl. 514/12; 514/183; 514/450; 514/675; 514/44 R

(57) ABSTRACT

The present invention generally provides therapeutic compositions and methods for treating a disease, disorder, or injury characterized by a deficiency in cell number. The method involves inducing a heat shock response in tissue or organ effected by disease and recruiting stem cells to repair or regenerate the disease-effected tissue.

### CROSS-REFERENCE TO RELATED APPLICATION

This application claims the benefit of U.S. Provisional Application No. 60/833,898, filed on Jul. 27, 2007, the entire contents of which are incorporated herein by reference.

### STATEMENT OF RIGHTS TO INVENTIONS MADE UNDER FEDERALLY SPONSORED RESEARCH

This work was supported by a National Eye Institute Grant, Grant No. EY016070-01. The government may have certain rights in the invention.

### BACKGROUND OF THE INVENTION

Methods for regenerating or repairing damaged tissues may be used to address a variety of diseases, disorders, and injuries characterized by a loss or a deficiency in a particular cell type. Such cell loss may be associated with trauma, ischemic injury, metabolic disease, or a degenerative disorder. Organ transplantation has conventionally been used to replace damaged or diseased tissues. Unfortunately, the supply of donor organs is limited. Even when donor organs are, available, rejection of the transplanted biological material can occur. Methods for promoting tissue repair and regeneration are urgently required.

### SUMMARY OF THE INVENTION

As described below, the present invention features methods of treating a disease or disorder characterized by a deficiency in cell number.

In one aspect, the invention features a method for ameliorating a disease (e.g., ischemic injury, myocardial infarction, muscle ischemia, a neural, glial, or muscle degenerative disorder, muscular atrophy or dystrophy, heart disease, congenital heart failure, hepatitis, cirrhosis of the liver, an autoimmune disorder, diabetes, cancer, a congenital defect that results in the absence of a tissue or organ, angina pectoris, myocardial infarction, ischemic limb, accidental tissue defect, fracture or wound) characterized by a deficiency in cell number in a subject. The method involves inducing heat shock in at least one cell of a tissue having a deficiency in cell number; and recruiting a stem cell (e.g., a hematopoietic stem cell) to the tissue (e.g., bladder, brain, nervous tissue, glia, esophagus, fallopian tube, heart, pancreas, intestines, gall bladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage), thereby ameliorating the disease. In one embodiment, the heat shock is induced in the tissue using sub-visible threshold laser (SVL) stimulation. In another embodiment, the heat shock is induced using a polypeptide (e.g., Hsp70 or Hsp90), nucleic acid molecule or a small compound. In another embodiment, the small compound is any one or more of geldanamycin, celastrol, 17-allylamino-17-demethoxygeldanamycin, ECl02, radicicol, geranylgeranylacetone, paeoniflorin, PU-DZ8, and H-71. In yet another embodiment, the method increases the expression or activity of a heat shock protein selected from the group consisting of Hsp100, Hsp90, Hsp70, Hsp60, and Hsp40. Preferably, the method reduces at least one symptom of the disease.

In another aspect, the invention provides a method of recruiting a stem cell to a tissue having a deficiency in cell number. The method involves stimulating the tissue with a sub-threshold laser, wherein the level of stimulation is sufficient to recruit at least one stem cell to the tissue. In one embodiment, a 15% duty cycle is used. In other embodiments, the sub-threshold laser has a wavelength from at least about 100 nm to 2000 nm (e.g., 100, 200, 300, 400, 500, 750, 1000, 1250, 1500, 1750, 2000). In another embodiment, the sub-threshold laser energy is from about 5 mW to 200 mW or is from about 10 mW to 100 mW. In one embodiment, the laser is administered in a micropulse. In other embodiments, the duration of the micropulse is from about 0.001 msec to 1.0 msec (e.g., 0.001, 0.002, 0.005, 0.01, 0.02, 0.05, 0.075, 0.1, 0.5, 0.75, 1.0 msec). In one embodiment, the duration of the micropulse is 0.1 msec. In other embodiments, the sub-threshold laser energy is between 10 mW to 100 mW and is administered in a 0.1 msec pulse. In still other embodiments, the stimulation increases the expression or biological activity of a heat shock protein selected from the group consisting of Hsp100, Hsp90, Hsp70, Hsp60, and Hsp40. In still other embodiments, the stimulation alters the expression or activity of a protein selected from the group consisting of SDF-1, VEGF, HIF-1α, crystallin, hypoxia-inducible factor 1-alpha (HIF-1a), and CXCR-4. In still other embodiments, the method increases the expression of an Hsp70 or Hsp90 polypeptide by at least 10-fold or by at least 40-fold.

In yet another aspect, the invention provides a method of recruiting a stem cell to a tissue of a subject in need thereof. The method involves administering a pharmacological agent to a subject in an amount sufficient to induce heat shock in the tissue; and recruiting a stem cell to the tissue.

In still another aspect, the invention provides a method of ameliorating a disease or disorder characterized by a deficiency in cell number in a subject in need thereof. The method involves administering a pharmacological agent to a subject in an amount sufficient to induce heat shock in the tissue; and recruiting a stem cell to the tissue, thereby ameliorating the disease or disorder.

In yet another aspect, the invention provides a method of regenerating a tissue in a subject in need thereof. The method involves administering a pharmacological agent to a subject in an amount sufficient to induce heat shock in a tissue; and recruiting a stem cell to the tissue, thereby regenerating the tissue.

In yet another aspect, the invention provides a method of repairing organ damage in a subject in need thereof. The method involves administering a pharmacological agent to a subject in an amount sufficient to induce heat shock in at least one cell of the organ; and recruiting a stem cell to the organ, thereby repairing the organ.

In yet another aspect, the invention provides a method of ameliorating a disease or disorder in a subject in need thereof. The method involves administering to the subject an agent that mobilizes a bone marrow derived stem cell in the subject; inducing heat shock in a tissue having a deficiency in cell number; and recruiting the stem cell to the tissue, thereby ameliorating the disease or disorder.

In yet another aspect, the invention provides a method of ameliorating cell loss in a tissue of a subject in need thereof. The method involves administering to the subject GM-CSF and/or Stem Cell Factor, wherein the administration mobilizes a bone marrow derived stem cell in the subject; inducing heat shock in a tissue by administering a subthreshold laser treatment or pharmacological agent; and recruiting the bone marrow derived stem cell to the tissue, thereby ameliorating the cell loss.

In yet another aspect, the invention provides a method for increasing SDF-1 expression in a cell (e.g., a human in vivo or in vitro), the method involves contacting the cell with an effective amount of celastrol, thereby increasing SDF-1 expression. In one embodiment, the cell is any one or more of a liver cell, skin cell, neural cell, lung cell, and brain cell. In another embodiment, the cell is present in a tissue or organ. In yet another embodiment, the increase in SDF-1 expression (e.g., a 5%, 10%, 25%, 50%, 75%, 100%, 200%, 300%, 400%, 500%, 5000%, 10,000%, 15,000%, or 20,000% increase) is sufficient to recruit a stem cell to the tissue or organ. In yet another embodiment, the stem cell recruitment repairs tissue damage. In yet another embodiment, the tissue damage is related to ischemic injury, myocardial infarction, neurodegeneration, wounding, cirrhosis, apoptotic or necrotic cell death.

In yet another aspect, the invention provides a pharmaceutical composition for recruiting stem cells to a tissue, the composition contains an effective amount of a small compound selected from the group consisting of geldanamycin, celastrol, 17-allylamino-17-demethoxygeldanamycin, ECl02, radicicol, geranylgeranylacetone, paeoniflorin, PU-DZ8, and H-71.

In yet another aspect, the invention provides a pharmaceutical composition for recruiting stem cells to a tissue, the composition contains an expression vector containing a polynucleotide encoding a heat shock polypeptide (e.g., Hsp100, Hsp90, Hsp70, Hsp60, and Hsp40) in a pharmaceutically acceptable excipient.

In yet another aspect, the invention provides a pharmaceutical composition for stem cell recruitment in a tissue, the composition contains a Hsp100, Hsp90, Hsp70, Hsp60, and Hsp40 polypeptide or polynucleotide in a pharmaceutically acceptable excipient.

In yet another aspect, the invention provides a kit containing an effective amount of an agent (e.g., a polypeptide, a polynucleotide, or a small compound) that induces a heat shock response in a tissue, and instructions for using the kit to increase stem cell recruitment in the tissue. In still other embodiments, the polypeptide is a heat shock polypeptide (e.g., Hsp100, Hsp90, Hsp70, Hsp60, and Hsp40). In still other embodiments, the polynucleotide encodes Hsp100, Hsp90, Hsp70, Hsp60, and Hsp40. In still other embodiments, the small compound is selected from the group consisting of geldanamycin, celastrol, 17-allylamino-17-demethoxygeldanamycin, ECl02, radicicol, geranylgeranylacetone, paeoniflorin, PU-DZ8, and H-71.

In yet another aspect, the invention provides a method of identifying an agent that increases stem cell recruitment in a tissue. The method involves contacting a cell of the tissue with a test compound; identifying an increase in the expression or activity of a heat shock polypeptide in the tissue relative to an untreated tissue, thereby identifying a compound that increases stem cell recruitment.

In yet another aspect, the invention provides a method of identifying an agent that increases stem cell recruitment in a tissue. The method involves contacting a cell of the tissue with a test compound; and identifying an increase in the number of stem cells in the tissue, thereby identifying a compound that increases stem cell recruitment.

In various embodiments of any of the above aspects, the subject (e.g., mammal, such as a human patient) has a disease related to excess or undesirable cell death or deficiencies in cell number. Such diseases include any one or more of the following: ischemic injury, myocardial infarction, muscle ischemia, a neural, glial, or muscle degenerative disorder, muscular atrophy or dystrophy, heart disease, congenital heart failure, hepatitis, cirrhosis of the liver, an autoimmune disorder, diabetes, cancer, a congenital defect that results in the absence of a tissue or organ, angina pectoris, myocardial infarction, ischemic limb, accidental tissue defect, fracture or wound. the agent is a polypeptide, a polynucleotide or a small compound. In other embodiments, the small compound is selected from the group consisting of geldanamycin, celastrol, 17-allylamino-17-demethoxygeldanamycin, ECl02, radicicol, geranylgeranylacetone, paeoniflorin, PU-DZ8, and H-71. In still other embodiments, the pharmacological agent is administered locally or systemically. In still other embodiments, the administration induces cellular repair or regeneration of the tissue. In still other embodiments, the stem cell contains a vector encoding a therapeutic polypeptide. In yet other embodiments, the method further involves locally or systemically administering an isolated stem cell to enhance tissue repair or regeneration. In various embodiments of any of the above aspects, the agent increases the expression or biological activity of a heat shock protein selected from the group consisting of Hsp100, Hsp90, Hsp70, Hsp60, and Hsp40. In various embodiments of any of the above aspects, the agent alters the expression or activity of a protein selected from the group consisting of SDF-1, VEGF, HIF-1α, crystallin, hypoxia-inducible factor 1-alpha (HIF-1a), and CXCR-4. In various embodiments of any of the above aspects, the method increases the expression of an Hsp70 or Hsp90 polypeptide by at least 10-fold or 40-fold. In still other embodiments of any of the above aspects, the method further involves administering to a subject an agent that increases hematopoietic stem cell mobilization is prior to induction of the heat shock response. In still other embodiments of any of the above aspects, the method further involves administering an anti-inflammatory agent or an anti-angiogenic agent. In still other embodiments of any of the above aspects, the method further involves administering an agent that supports the survival, proliferation, or transdifferentiation of a hematopoietic stem cell. In still other embodiments of any of the above aspects, the agent is granulocyte macrophage colony stimulating factor or stem cell factor. In still other embodiments of any of the above aspects, the heat shock is induced using a subthreshold laser treatment. In still other embodiments of any of the above aspects, the heat shock is induced using an agent (e.g., polypeptide, nucleic acid molecule, small compound). A small compound is any one or more of the following geldanamycin, celastrol, 17-allylamino-17-demethoxygeldanamycin, ECl02, radicicol, geranylgeranylacetone, paeoniflorin, PU-DZ8, and H-71. In various embodiments of any of the above aspects, the cell is not an ocular cell, retinal cell, retinal epithelial cell, or other cell of the eye.

The invention provides methods of treating diseases characterized by a deficiency in cell number. Other features and advantages of the invention will be apparent from the detailed description, and from the claims.

### DEFINITIONS

By “agent” is meant any small molecule chemical compound, antibody, nucleic acid molecule, or polypeptide, or fragments thereof.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

By “analog” is meant a structurally related polypeptide or nucleic acid molecule having the function of a reference polypeptide or nucleic acid molecule.

By “biological activity of a heat shock protein” is meant a chaperone activity or stem cell recruiting activity.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. patent law and can mean “includes,” “including,” and the like; “consisting essentially” of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “deficiency in cell number” is meant fewer of a specific set of cells than are normally present in a tissue or organ not having a deficiency. For example, a deficiency is a 5%, 10%, 15%, 20%, 30%, 40%, 50%, 60%, 70%, 80%, 90%, or even 100% deficit in the number of cells of a particular cell-type relative to the number of cells present in a naturally-occurring, corresponding tissue or organ. Methods for assaying cell-number are standard in the art, and are described in (Bonifacino et al., Current Protocols in Cell Biology, Loose-leaf, John Wiley and Sons, Inc., San Francisco, Calif., 1999; Robinson et al., Current Protocols in Cytometry Loose-leaf, John Wiley and Sons, Inc., San Francisco, Calif., October 1997).

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “labeled nucleic acid or polypeptide” is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

By “expression vector” is meant a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “heat shock” is meant any cellular response to thermal stress. Typically, cells respond to heat shock by increasing the transcription or translation of a heat shock polypeptide (e.g., Hsp70 or 90).

By “heat shock polypeptide” is meant any polypeptide expressed in a cell in response to thermal stress. Exemplary heat shock polypeptides include, but are not limited to, Hsp100, Hsp90, Hsp70, Hsp60, and Hsp40. An exemplary Hsp70 amino acid sequence is provided at GenBank Accession No. AAA02807. Exemplary Hsp90 amino acid sequence is provided at GenBank Accession Nos. P08238, NP_{—}005339, NP_{—}001017963, and P07900.

By “heat shock response activator” is meant a compound that increases the chaperone activity or expression of a heat shock pathway component. Heat shock pathway components include, but are not limited to, Hsp100, Hsp90, Hsp70, Hsp60, Hsp40 and small HSP family members. Agents or treatments that induce heat shock typically increase the expression or activity of at least one of Hsp70 or Hsp90.

By “hematopoietic stem cell” is meant a bone marrow derived cell capable of giving rise to one or more differentiated cells of the hematopoietic lineage.

By “hematopoietic stem cell mobilization” is meant increasing the number of bone marrow derived stem cells available for recruitment to an organ or tissue in need thereof.

By “ocular disease or disorder” is meant a pathology effecting the normal function of the eye.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “polypeptide” is meant any chain of amino acids, regardless of length or post-translational modification.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “promoter” is meant a polynucleotide sufficient to direct transcription. Exemplary promoters include nucleic acid sequences of lengths 100, 250, 300, 400, 500, 750, 900, 1000, 1250, and 1500 nucleotides that are upstream (e.g., immediately upstream) of the translation start site.

By “recruit” is meant attract for incorporation into a tissue.

By “reduces” or “increases” is meant a negative or positive alteration, respectively, of at least 10%, 25%, 50%, 75%, or 100%.

By “regenerating a tissue” is meant increasing the number, survival, or proliferation of cells in the tissue.

By “repairing tissue damage” is meant ameliorating cell injury, damage, or cell death.

By “stem cell” is meant a progenitor cell capable of giving rise to one or more differentiated cell types.

By “subject” is meant a mammal, including, but not limited to, a human or non-human mammal, such as a bovine, equine, canine, ovine, or feline.

By “subthreshold laser” is meant a laser therapy that induces a lesion that is undetectable or barely detectable in the tissue during or following treatment. A lesion is “undetectable” where little or no intraoperative visible tissue reaction is present or where little or no cell death (e.g., less than 10%, 5%, 2.5%, 1% of cells in treated tissue die or apoptose) due to laser treatment.

By “tissue” is meant a collection of cells having a similar morphology and function.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

By “transdifferentiation” is meant altering the cell, such that it expresses at least one polypeptide characteristically expressed by a cell of a different type.

### BRIEF DESCRIPTION OF THE DRAWINGS

FIG. 1 shows a series of ocular tissue mounts. The dark regions in FIG. 1 represent GFP⁺ cells that have incorporated into the RPE layer in areas that have received laser. Background fluorescence, as determined by the contralateral (unaffected) eye, was removed.

FIG. 2 is a graph quantitating human stem cell (HSC) incorporation into the retinal pigment epithelium (RPE).

FIG. 3 is a series of panels showing ocular tissue mounts taken from mice that received suborbital injection of GFP⁺ HSC in combination with pharmacologically induced heat shock using geldanamycin derivatives D28 or H71; that received HSP70 polypeptide injection.

FIGS. 4A, 4B, and 4C are graphs showing that mice injected periotoneally with the chemical heat shock inducer, celastrol, showed an increase in expression of the SDF-1, the most potent chemoattractant for endogenous stem cells known, in a variety of tissues relative to SDF-1 expression in a control mouse injected with a placebo. This was measured as percent SDF-1/TBP. “PC” denotes posterior eyecup (i.e. the retinal pigment epithelial cells); “P1” denotes the control condition; “T1,” “T2,” “T3” and “T4” denote one, two, three, and four days post-infection, respectively.

### DETAILED DESCRIPTION OF THE INVENTION

The present invention generally provides therapeutic compositions and methods for treating a disease, disorder, or injury characterized by a deficiency in cell number. The method involves inducing a heat shock response in tissue or organ effected by disease and recruiting stem cells to repair or regenerate the disease-effected tissue.

While the examples specifically describe the use of heat shock response induction in the retinal pigment epithelial layer and the choroid, the invention is not so limited. Induction of the heat shock response can be used to enhance stem cell recruitment to virtually any cell or tissue. Accordingly, the invention provides compositions comprising heat shock response activators useful for recruiting stem cells to a tissue or organ having a deficiency in cell number. Induction of a heat shock response in at least one cell of the tissue increases stem cell recruitment and provides for the repair or regeneration of the tissue or organ. Such methods are useful for treating subjects having a deficiency in a particular cell type.

### Hematopoietic Stem Cells

Hematopoietic stem cells are bone marrow-derived cells that represent an endogenous source known for their reparative potential as well as for their plasticity. Bone marrow-derived hematopoietic stem cells (HSCs) are able to repair damaged tissues, including heart, liver, brain, muscle and kidney.

Stem cells are recruited to areas of injury to effect the repair or regeneration of the injured tissue. If desired, the number of hematopoietic stem cells present in the circulation of a subject may be increased prior to, during, or following induction of heat shock. In one embodiment, this increase in hematopoietic stem cell number is accomplished by mobilizing hematopoietic stem cells present in the bone marrow of the subject by administering any one or more of granulocyte-macrophage colony stimulating factor (G-CSF), stem cell factor (SCF), IL-8, SDF-1 (stromal derived factor), interleukin-1 (IL-1), interleukin-3 (IL-3), interleukin-6 (IL-6), interleukin-7 (IL-7), interleukin-8 (IL-8), interleukin-11 (IL-11), interleukin-12 (IL-12), and NIP-1α, stem cell factor (SCF), fims-like tyrosine kinase-3 (flt-3), transforming growth factor-β (TGF-β), an early acting hematopoietic factor, described, for example in WO 91/05795, and thrombopoietin (Tpo), FLK-2 ligand, FLT-2 ligand, Epo, Oncostatin M, and MCSF. SDF-1 is a potent cytokine that induces the recruitment of stem cells. SDF-1 is expressed by RPE cells during stress. Administration of G-CSF and/or SDF-1 will increase the number of HSC in the peripheral blood and will likely enhance subsequent HSC recruitment to a damaged or diseased tissue or organ. Preferably, hematopoietic stem cells of the invention fail to express or express reduced levels of any one or more of the following markers: Lin⁻, CD2⁻, CD3⁻, CD7⁻, CD8⁻, CD10⁻, CD14⁻, CD15⁻, CD16⁻, CD19⁻, CD20⁻, CD33⁻, CD38⁻, CD71⁻, HLA-DR⁻, and glycophorin A⁻.

### Lasers

Ophthalmic lasers are an important tool for the treatment of various retinal disorders where they have typically been used to generate laser-induced photochemical burns. In contrast, the diode 810 nanometer laser is believed to cause less damage to the neurosensory retina because the energy is absorbed by the retinal pigmented epithelium. The present invention provides methods for using a subvisible laser application to mobilize hematopoietic stem cells and recruit them to a tissue of interest. In the present method, an infrared (810 nm) laser is used in micropulse mode for the treatment of diseases or disorder characterized by a deficiency in cell number. By using repetitive, brief pulses of laser during a single exposure, it limits the amount of heat conduction and subsequent cellular damage within the stimulated tissue. In one approach, laser administration is controlled to reduce or eliminate photothermal damage. For example, the laser treatment is controlled to reduce or eliminate intraoperative visible tissue reaction and or late cellular death (apoptosis). In other examples, the threshold of non-lethal thermal injury is controlled such that intraoperative visible tissue reaction is reduced or absent, late cellular death is reduced or absent, and consistent positive HSC recruitment is present. Preferably, the photothermal damage is reduced by at least 10%, 25%, or 30% relative to a patient treated with conventional laser therapy; more preferably, photothermal damage is reduced by at least 50%, 75%, 85%, 95% or 100%.

Methods of inducing heat shock using a sub-threshold laser include, for example, administering a grid pattern of 40-50 well-spaced 810 nm-laser spots with a diameter of 5 μm, 10 μm, 25 μm, 35 μm, or 50 μm to the tissue of interest. The power and delivery modalities may be varied to reduce or eliminate photothermal damage. For example, a continuous-wave (cw) delivery mode; a microPulse (mP) delivery mode (e.g., using 20%, 15%, 10% and 5% duty cycle); or a long pulse delivery mode may be used.

In particular embodiments, the present methods feature the use of a sub-threshold laser having a wavelength from at least about 100 nm up to 2000 nm, where the sub-threshold laser energy is at least about 10 mW to 100 mW (e.g., 10, 20, 30, 40, 50, 60, 70, 80, 90 or 100). The laser is applied for a duration of at least 0.001, 0.005, 0.1, 0.2 or 1.0 msec. In other embodiments, 10 mW is administered in a 0.1 msec pulse or 100 mW is administered in a 0.1 msec pulse.

Tissues amenable to laser treatment include not only epithelial tissues that are widely accessible to topical therapy using a laser, but also tissues that can be accessed surgically. Preferably, the tissue or organ is exposed in a surgical procedure and stimulated using a laser to induce heat shock in at least one cell of the tissue where stem cell repair or regeneration is desired. While specific examples described herein relate to the use of lasers to induce heat shock, one skilled in the art appreciates that virtually any method of energy delivery capable of inducing the heat shock response in at least one cell of a tissue may be used. Such methods include, for example, stimulation using radiation, transpupillary thermography or any other form of energy, such as light energy, in an amount sufficient to stimulate stem cell recruitment. In various embodiments, laser stimulation sufficient to recruit stem cells refers to a light beam, or photons that have a wavelength of from about 100 nm up to 2000 nm. Usually the wavelength is between about 500 nm to about 900 nm.

### Heat Shock Response Activators

Heat shock induction can also be achieved using pharmacological agents. Heat shock response activators include agents (e.g., small compound, polypeptide, and nucleic acid molecules) that induce a heat shock response in a cell. Such agents increase, for example, the expression of biological activity of a heat shock protein, such as Hsp100, Hsp90, Hsp70, Hsp60, Hsp40 and small HSP family members. More preferably, the agent increases the expression or biological activity of Hsp90 or Hsp70. Heat shock protein 90 (Hsp90) is a chaperone involved in cell signaling, proliferation and survival, and is essential for the conformational stability and function of a number of proteins. HSP-90 modulators are useful in the methods of the invention, such modulators increase the expression or the biological activity of a HSP90. HSP90 modulators include benzoquinone ansamycin antibiotics, such as geldanamycin and 17-allylamino-17-demethoxygeldanamycin (17-AAG), which specifically bind to Hsp90, and alter its function. Other Hsp90 modulators include, but are not limited to, radicicol, novobiocin, and any Hsp90 inhibitor that binds to the Hsp90 ATP/ADP pocket.

Other agents that induce heat shock include, but are not limited to, geldanamycin, celastrol, 17-allylamino-17-demethoxygeldanamycin, ECl02, radicicol, geranylgeranylacetone, paeoniflorin, PU-DZ8, and H-71. Celastrol, a quinone methide triterpene, activates the human heat shock response. Celastrol and other heat shock response activators are useful for the treatment of diseases associated with a deficiency in cell number. Heat shock response activators include, but are not limited to, celastrol, celastrol methyl ester, dihydrocelastrol diacetate, celastrol butyl ester, dihydrocelastrol, and salts or analogs thereof.

### Diseases Characterized by a Deficiency in Cell Number

The invention may be used for the treatment of virtually any disease associated with a deficiency in cell number. The deficiency in cell number may be associated with undesirable cell death to disease or ischemic injury. For example, the mammal may have a disease, disorder, or condition that results in the loss, atrophy, dysfunction, or death of cells. Exemplary treated conditions include a neural, glial, or muscle degenerative disorder, muscular atrophy or dystrophy, heart disease, such as congenital heart failure, hepatitis or cirrhosis of the liver, an autoimmune disorder, diabetes, cancer, a congenital defect that results in the absence of a tissue or organ, or a disease, disorder, or condition that requires the removal of a tissue or organ, ischemic diseases, such as angina pectoris, myocardial infarction and ischemic limb, accidental tissue defect or damage such as fracture or wound. In other embodiments, the mammal has an increased risk of developing a disease, disorder, or condition that is delayed or prevented by the method.

In various embodiments, the tissue or organ is selected from the group consisting of bladder, brain, nervous tissue, glia, esophagus, fallopian tube, heart, pancreas, intestines, gall bladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. Preferably, the recruitment of stem cells increases the biological function of a diseased or damaged tissue or organ by at least 5%, 10%, 25%, 50%, 75%, 100%, 200%, or even by as much as 300%, 400%, or 500%. In other preferred embodiments, the method increases the number of cells of the tissue or organ by at least 5%, 10%, 20%, more desirably by at least 25%, 30%, 35%, 40%, 50%, 60%, or even by as much as 70%, 80%, 90 or 100% compared to a corresponding tissue or organ. Methods for assaying cell number, survival or proliferation are known to the skilled artisan and are described in (Bonifacino et al., Current Protocols in Cell Biology Loose-leaf, John Wiley and Sons, Inc., San Francisco, Calif.).)

Stem cells of the invention are recruited to the tissue or organ in need of repair, where they transdifferentiate. Stem cells of the invention transdifferentiate to skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes. In preferred embodiments, the method increases the biological activity of the tissue or organ by at least 5%, 10%, 20%, 30%, 40%, 50%, 60%, 70%, 80%, 90%, 100%, 150%, or even by as much as 200%, 300%, 400%, or 500% compared to a corresponding, naturally-occurring tissue or organ. Biological functions of the tissue or organ amenable to assay include digestion, excretion of waste, secretion, electrical activity, muscle activity, hormone production, or other metabolic activity. Methods for assaying the biological function of virtually any organ are routine, and are known to the skilled artisan (e.g., Guyton et al., Textbook of Medical Physiology, Tenth edition, W.B. Saunders Co., 2000).

Methods of the invention are useful for treating or stabilizing in a patient (e.g., a human or mammal) a condition, disease, or disorder affecting a tissue or organ. Therapeutic efficacy is optionally assayed by measuring, for example, the biological function of the treated tissue or organ (e.g., bladder, bone, brain, breast, cartilage, esophagus, fallopian tube, heart, pancreas, intestines, gallbladder, kidney, liver, lung, nervous tissue, ovaries, prostate, skeletal muscle, skin, spinal cord, spleen, stomach, testes, thymus, thyroid, trachea, ureter, urethra, urogenital tract, and uterus). Such methods are standard in the art. For example, bladder function is assayed by measuring urine retention and excretion. Brain, spinal cord, or nervous tissue function is assayed by measuring neural activity (e.g., electrical activity). Esophageal function is assayed by measuring the ability of the esophagus to convey food to the stomach. Heart function is assayed by electrocardiogram. Pancreatic function is assayed by measuring insulin production. Intestinal function is assayed by measuring the ability of intestinal contents to pass through to the bowel, and may be evaluated using a barium enema or gastrointestinal series. Gallbladder function is assayed using a gall bladder radionuclide scan. Kidney function is assayed by measuring creatinine levels, urine creatinine levels, or by clinical tests for creatinine clearance, or blood urea nitrogen. Liver function is assayed using liver function tests or a liver panel that measures liver enzyme levels, bilirubin levels, and albumin levels. Lung function is assayed using spirometry, lung volume, and diffusion capacity tests. Ovary function is assayed by measuring levels of ovarian hormones (e.g., follicle stimulating hormone). Prostate abnormality is assayed by measuring prostate specific antigen. Spleen function is assayed using a liver-spleen scan. Stomach function is assayed using a stomach acid test or by assaying gastric emptying. Testicular function is assayed by measuring levels of testicular hormones (e.g., testosterone). Other methods for assaying organ function are known to the skilled artisan and are described, for example, in the Textbook of Medical Physiology, Tenth edition, (Guyton et al., W.B. Saunders Co., 2000).

### Screening Assays

As discussed herein, compounds that induce heat shock or stem cell recruitment to a tissue having a deficiency in cell number are useful in the methods of the invention. Any number of methods are available for carrying out screening assays to identify such compounds. In one approach, the expression of an HSP polypeptide or nucleic acid molecule is monitored in a cell (e.g., a cell in vitro or in vivo); the cell is contacted with a candidate compound; and the effect of the compound on HSP polypeptide or nucleic acid molecule expression is assayed using any method known in the art or described herein. A compound that increases the expression of an HSP polypeptide or nucleic acid molecule in the contacted cell relative to a control cell that was not contacted with the compound, is considered useful in the methods of the invention. Alternatively, compounds are screened to identify those that increase stem cell recruitment to the retina. In one embodiment, stem cell recruitment is assayed in a chimeric mouse injected locally or systemically with GFP⁺ expressing stem cells. The presence of GFP⁺ cells is assayed, for example, by examining retinal flat mounts using fluorescence microscopy. Compounds that the number of stem cells recruited to the retina are useful in the methods of the invention. In other embodiments, the survival or differentiation of such cells is assayed using cell specific markers. In a related approach, the screen is carried out in the presence of 11-cis-retinal, 9-cis-retinal, or an analog or derivative thereof. Useful compounds increase the number of stem cells recruited to the retina by at least 10%, 15%, or 20%, or preferably by 25%, 50%, or 75%; or most preferably by at least 100%.

If desired, the efficacy of the identified compound is assayed in an animal model having a disease (e.g., an animal model of having a deficiency in cell number caused, for example, cell death).

### Test Compounds and Extracts

In general, compounds capable of inducing a heat shock response in a cell or increasing stem cell recruitment to an ocular tissue are identified from large libraries of either natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in recruiting stem cells or inducing heat shock should be employed whenever possible.

When a crude extract is found to recruit stem cells or induce heat shock further fractionation of the positive lead extract is necessary to isolate chemical constituents responsible for the observed effect. Thus, the goal of the extraction, fractionation, and purification process is the careful characterization and identification of a chemical entity within the crude extract that induce heat shock or stem cell recruitment. Methods of fractionation and purification of such heterogenous extracts are known in the art. If desired, compounds shown to be useful agents for the treatment of any pathology related to an ocular disease requiring the repair or regeneration of an ocular tissue are chemically modified according to methods known in the art.

### Pharmaceutical Compositions

The present invention features pharmaceutical preparations comprising agents capable of inducing heat shock in a tissue or organ together with pharmaceutically acceptable carriers. Such preparations have both therapeutic and prophylactic applications. Agents useful in the methods described herein include those that increase the expression or biological activity of an Hsp90 polypeptide, or HSP70, or that otherwise induce a heat shock response in a tissue thereby recruiting a stem cell to the tissue. If desired, the compositions of the invention are formulated together with agents that increase the number of hematopoietic stem cells present in the circulation of a subject, for example, by mobilizing hematopoietic stem cells present in the bone marrow of the subject.

Agents that increase the mobilization or recruitment of stem cells include, but are not limited to, antiblastic drugs and G-CSF or GM-CSF, interleukin-1 (IL-1), interleukin-3 (IL-3), interleukin-6 (IL-6), interleukin-7 (IL-7), interleukin-8 (IL-8), interleukin-11 (IL-11), interleukin-12 (IL-12), and NIP-1α, stem cell factor (SCF), fims-like tyrosine kinase-3 (flt-3), transforming growth factor-β (TGF-β.), an early acting hematopoietic factor, described, for example in WO 91/05795, and thrombopoietin (Tpo), FLK-2 ligand, FLT-2 ligand, Epo, Oncostatin M, and MCSF.

Compounds of the invention may be administered as part of a pharmaceutical composition. The compositions should be sterile and contain a therapeutically effective amount of the agents of the invention in a unit of weight or volume suitable for administration to a subject. The compositions and combinations of the invention can be part of a pharmaceutical pack, where each of the compounds is present in individual dosage amounts.

Pharmaceutical compositions of the invention to be used for prophylactic or therapeutic administration should be sterile. Sterility is readily accomplished by filtration through sterile filtration membranes (e.g., 0.2 μm membranes), by gamma irradiation, or any other suitable means known to those skilled in the art. Therapeutic polypeptide compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution.

The compounds may be combined, optionally, with a pharmaceutically acceptable excipient. The term “pharmaceutically-acceptable excipient” as used herein means one or more compatible solid or liquid filler, diluents or encapsulating substances that are suitable for administration into a human. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be also included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having a disease or disorder characterized by a deficiency in cell number, an effective amount is sufficient to induce heat shock in at least one cell of the tissue; sufficient to attract at least one stem cell to the tissue; or sufficient to stabilize, slow, or reduce a symptom associated with a pathology. Generally, doses of the compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of a composition of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In one preferred embodiment, a composition of the invention is administered intraocularly. Other modes of administration include oral, rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Compositions comprising a composition of the invention can be added to a physiological fluid, such as to the intravitreal humor. Oral administration can be preferred for prophylactic treatment because of the convenience to the patient as well as the dosing schedule.

Pharmaceutical compositions of the invention can optionally further contain one or more additional proteins as desired. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian protein which has been introduced according to standard recombinant DNA techniques; or from the human biological fluids (e.g., blood, milk, lymph, urine or the like) or from transgenic animals that contain a gene that expresses a human protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g, tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising a compound of the present invention can contain multivalent metal ions, such as calcium ions, magnesium ions and/or manganese ions. Any multivalent metal ion that helps stabilizes the composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the compound contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly (2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Compositions of the invention may also be delivered topically. For topical delivery, the compositions are provided in any pharmaceutically acceptable excipient that is approved for ocular delivery. Preferably, the composition is delivered in drop form to the surface of the eye. For some application, the delivery of the composition relies on the diffusion of the compounds through the cornea to the interior of the eye.

Those of skill in the art will recognize that the best treatment regimens for using compounds of the present invention to treat an ocular disease can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### Combination Therapies

Compositions and methods of the invention may be administered in combination with any standard therapy known in the art. If desired, an agent that induces heat shock in a tissue or a subthreshold laser regimen (described herein) is administered together with an agent that promotes the recruitment, survival, proliferation or transdifferentiation of a stem cell (e.g., a hematopoietic stem cell). Such agents include collagens, fibronectins, laminins, integrins, angiogenic factors, anti-inflammatory factors, glycosaminoglycans, vitrogen, antibodies and fragments thereof, functional equivalents of these agents, and combinations thereof.

In other embodiments, an agent that induces heat shock in a tissue or a subthreshold laser regimen (described herein) of the invention is administered in combination with an anti-inflammatory compound that is conventionally administered for the treatment of a disease. Such anti-inflammatory compounds include, but are not limited to, any one or more of steroidal and non-steroidal compounds and examples include: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; or Zomepirac Sodium.

In still other embodiments, an agent that induces heat shock in a tissue or a subthreshold laser regimen of the invention is administered in combination with an agent that increases or modulates angiogenesis in the tissue. Such agents are capable of modulating the expression or activity of an angiogenic factor, such as platelet derived growth factor (PDGF), vascular endothelial growth factor (VEGF), basic fibroblast growth factor (bFGF), bFGF-2, leptins, plasminogen activators (tPA, uPA), angiopoietins, lipoprotein A, transforming growth factor-β, bradykinin, angiogenic oligosaccharides (e.g., hyaluronan, heparan sulphate), thrombospondin, hepatocyte growth factor (also known as scatter factor) and members of the CXC chemokine receptor family.

In other embodiments, agents and methods are administered together with chemotherapeutic agents that enhance bone marrow-derived stem cell mobilization, including cytoxan, cyclophosphamide, VP-16, and cytokines such as GM-CSF, G-CSF or combinations thereof.

Combinations of the invention may be administered concurrently or within a few hours, days, or weeks of one another. In one approach, an agent that induces heat shock in a tissue or a subthreshold laser regimen (described herein) is administered prior to, concurrently with, or following administration of a conventional therapeutic described herein. In some embodiments, it may be desirable to mobilize a bone marrow-derived cell prior to the induction of heat shock, where such mobilization increases the number of stem cells recruited to the tissue. In other embodiments, it may be preferable to administer the agent that mobilizes a bone marrow-derived cell concurrently with or following (e.g., within 1, 2, 3, 5 or 10 hours) of inducing heat shock.

### Methods for Increasing Stem Cell Recruitment to a Tissue

As reported herein, the induction of heat shock in a tissue effectively recruits stem cells (e.g., hematopoietic stem cells) to that tissue, where they ameliorate a disease or disorder. If desired, substantially purified stem cells (or their precursor or other progenitor cells) are administered to the patient in conjunction with an agent or treatment regimen (e.g., sub-threshold laser treatment) that induces heat shock in a tissue to facilitate the repair of the tissue. Preferably, the administered stern cells are from the same subject. In other embodiments, the stem cells are obtained from a donor. Such methods may be used to enhance the repair of a tissue by increasing the recruitment of stem cells to that tissue.

Methods of isolating hematopoietic stem cells are known in the art. In one embodiment, hematopoietic stem cells are isolated from the blood using apheresis. Apheresis for total white cells begins when the total white cell count is about 500-2000 cells/μl and the platelet count is about 50,000/μl. Daily leukapheris samples may be monitored for the presence of CD34⁺ and/or Thy-1⁺ cells to determine the peak of stem cell mobilization and, hence, the optimal time for harvesting peripheral blood stem cells. Various techniques may be employed to separate the cells by initially removing cells of dedicated lineage (“lineage-committed” cells), if desired. Monoclonal antibodies are particularly useful for identifying markers associated with particular cell lineages and/or stages of differentiation. The antibodies may be attached to a solid support to allow for crude separation. The separation techniques employed should maximize the viability of the fraction to be collected.

The use of separation techniques include those based on differences in physical properties (e.g., density gradient centrifugation and counter-flow centrifugal elutriation), cell surface properties (lectin and antibody affinity), and vital staining properties (mitochondria-binding dye rhodamine 123 and DNA-binding dye Hoechst 33342). Other procedures for separation that may be used include magnetic separation, using antibody-coated magnetic beads, affinity chromatography, cytotoxic agents joined to a monoclonal antibody or used in conjunction with a monoclonal antibody, including complement and cytotoxins, and “panning” with antibody attached to a solid matrix or any other convenient technique. Techniques providing accurate separation include flow cytometry (e.g., flow cytometry using a plurality of color channels, low angle and obtuse light scattering detecting channels, impedance channels).

A large proportion of differentiated cells may be removed from a sample using a relatively crude separation, where major cell population lineages of the hematopoietic system, such as lymphocytic and myelomonocytic, are removed, as well as lymphocytic populations, such as megakaryocytic, mast cells, eosinophils and basophils. Usually, at least about 70 to 90 percent of the hematopoietic cells will be removed.

Concomitantly or subsequent to a gross separation providing for positive selection, e.g. using the CD34 marker, a negative selection may be carried out, where antibodies to lineage-specific markers present on dedicated cells are employed. For the most part, these markers include CD2⁻, CD3⁻, CD7⁻, CD8⁻, CD10⁻, CD14⁻, CD15⁻, CD16⁻, CD19⁻, CD20⁻, CD33⁻, CD38⁻, CD71⁻, HLA-DR⁻, and glycophorin A; preferably including at least CD2⁻, CD14⁻, CD15⁻, CD16⁻, CD19⁻ and glycophorin A; and normally including at least CD14⁻ and CD15⁻. As used herein, Lin⁻ refers to a cell population lacking at least one lineage specific marker.

The purified stem cells have low side scatter and low to medium forward scatter profiles by FACS analysis. Cytospin preparations show the enriched stem cells to have a size between mature lymphoid cells and mature granulocytes. Cells may be selected based on light-scatter properties as well as their expression of various cell surface antigens.

Preferably, cells are initially separated by a coarse separation, followed by a fine separation, with positive selection of a marker associated with stem cells and negative selection for markers associated with lineage committed cells. Compositions highly enriched in stem cells may be achieved in this manner.

Purified or partiall purified stern cells are then administered to the patient. Administration may be local (e.g., by direct administration to the vitreous humor or to a vessel supplying a tissue of interest) or may be systemic.

### Polynucleotide Therapy to Induce Heat Shock

Polynucleotide therapy featuring a polynucleotide encoding an HSP protein, variant, or fragment thereof or a protein capable of activating heat shock is another therapeutic approach for treating a disease. Alternatively, the polynucleotides encode therapeutic polypeptides that enhance stem cell recruitment, survival, proliferation, or differentiation or otherwise ameliorate a symptom associated with the disease (e.g., reduce inflammation, angiogenesis, or cell death). Such polynucleotides can be delivered to cells of a subject having a disease where expression of the recombinant proteins will have a therapeutic effect. For example, nucleic acid molecules that encode therapeutic polypeptides are delivered to stem cells, such as bone marrow-derived stem cells, hematopoietic stem cells, their precursors, or progenitors. In other approaches, nucleic acid molecules are delivered to cells of a tissue. The nucleic acid molecules must be delivered to the cells of a subject in a form in which they can be taken up so that therapeutically effective levels of the therapeutic polypeptide (e.g., HSP protein, such as HSP 70, HSP 90) or fragment thereof can be produced.

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

If desired, a vector expressing stem cell recruiting factors is administered to a tissue or organ. SDF-1 (also called PBSF) (Campbell et al. (1998) Science 279(5349):381-4), 6-C-kine (also called Exodus-2), and MIP-3β (also called ELC or Exodus-3) induced adhesion of most circulating lymphocytes, including most CD4⁺ T cells; and MIP-3α (also called LARC or Exodus-1) triggered adhesion of memory, but not naive, CD4⁺ T cells. Tangemann et al. (1998) J. Immunol. 161:6330-7 disclose the role of secondary lymphoid-tissue chemokine (SLC), a high endothelial venule (HEV)-associated chemokine, with the homing of lymphocytes to secondary lymphoid organs. Campbell et al. (1998). J. Cell Biol 141(4):1053-9 describe the receptor for SLC as CCR7, and that its ligand, SLC, can trigger rapid integrin-dependent arrest of lymphocytes rolling under physiological shear.

In other approaches, vectors expressing anti-angiogenic polypeptides are administered to reduce vascularization in a tissue. Such anti-angiogenic polypeptides include, but are not limited to, interferon α, interferon β.

In still other approaches, a vector encoding a polypeptide characteristically expressed in a cell of interest is introduced to a stem cell of the invention.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an HSP protein, variant, or a fragment thereof, can be cloned into a retroviral vector and expression can be driven from its endogenous promoter, from the retroviral long terminal repeat, or from a promoter specific for a tissue or cell of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer an HSP polynucleotide into the eye.

Non-viral approaches can also be employed for the introduction of a therapeutic to a cell of a patient (e.g., a cell or tissue). For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine. Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTR) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified stem cell and/or in a cell of the tissue having a deficiency in cell number. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector preferably includes a selection gene, for example, a neomycin resistance gene, for facilitating selection of stem cells that have been transfected or transduced with the expression vector.

If desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant HSP protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Therapeutic Methods

The invention provides for the treatment of diseases and disorders associated with a deficiency in cell number. Many diseases associated with a deficiency in cell number are characterized by an increase in cell death. Such diseases include, but are not limited to, neurodegenerative disorders, stroke, myocardial infarction, or ischemic injury. Injuries associated with trauma can also result in a deficiency in cell number in the area sustaining the injury. Methods of the invention ameliorate such diseases, disorders, or injuries by generating cells that can supplement the deficiency. Such cells are generated from the recruitment and transdifferentiation of a cell to a cell type of interest (e.g., the reprogramming of a hematopoietic cell to cell) or by promoting the regeneration of a cell, tissue, or organ.

In one embodiment, an agent of the invention is administered to a cell, tissue, or organ in situ to induce heat shock, recruit stem cells, and repair or regenerate the tissue, such that an increase in cell number is achieved. Alternatively, hematopoietic stem cells are locally or systemically administered to a subject prior to, concurrent with, or subsequent to the induction of heat shock in a tissue or organ of the patient to enhance stem cell recruitment and ameliorate the disease, disorder, or injury.

Administration may be my any means sufficient to result in a sufficient level of stem cell recruitment. While the particular level of stem cell recruitment will vary depending on the therapeutic objective to be achieved, desirably at least 1, 2, 5, 10, or 15% of the cells present in the tissue are recruited stem cells after treatment. In other embodiments, at least 25%, 35%, or 50% of cells are recruited stem cells.

In various embodiments, agents of the invention are administered by local injection to a site of disease or injury, by sustained infusion, or by micro-injection under surgical conditions (Wolff et al., Science 247:1465, 1990). In other embodiments, the agents are administered systemically to a tissue or organ of a patient having a deficiency in cell number that can be ameliorated by cell regeneration.

The present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a disease or disorder or symptom thereof characterized by a deficiency in cell number. The method includes the step of administering to the mammal a therapeutic amount of an amount of a composition of the invention sufficient to treat the disease or disorder or symptom thereof, under conditions such that the disease or disorder is treated.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compositions herein may be also used in the treatment of any other disorders in which a deficiency in cell number may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a deficiency in cell number, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Kits

The invention provides kits for the treatment or prevention of a disease, disorder, or symptoms thereof associated with a deficiency in cell number. In one embodiment, the kit includes a pharmaceutical pack comprising an effective amount of a Hsp90 chaperone modulator (e.g., Geldanamycin and derivatives thereof) or a heat shock response activator (e.g., Celastrol). Preferably, the compositions are present in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject having or at risk of developing a disease or disorder associated with a deficiency in cell number. The instructions will generally include information about the use of the compounds for the treatment or prevention of a disease or disorder associated with a decrease in cell number. In other embodiments, the instructions include at least one of the following: description of the compound or combination of compounds; dosage schedule and administration for treatment of an ocular disorder or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

### Example 1

### Recruitment of Stem Cells by Laser

Chimeric mice were constructed with GFP⁺ stems cells that were transplanted into mice that had undergone near lethal irradiation. Chimeric mice were made with gfp-expressing hematopoietic stem cells from gfp homozygous transgenic donors. These cells were transplanted into recipients that had undergone near lethal irradiation. These gfp chimeric mice (C57B16.gfp) were used in all subsequent laser studies. Using the 810 nm diode laser with a spot diameter of 75 μm, various levels of energy were delivered to the retina including energy of 5 mJ (50 mW, 0.1 msec) that did not produce visible laser tissue reaction in the retina (laser irradiance 1130 W/cm²). This was considered sub-visible threshold. Three weeks post-laser the animals were euthanized and the eyes were harvested.

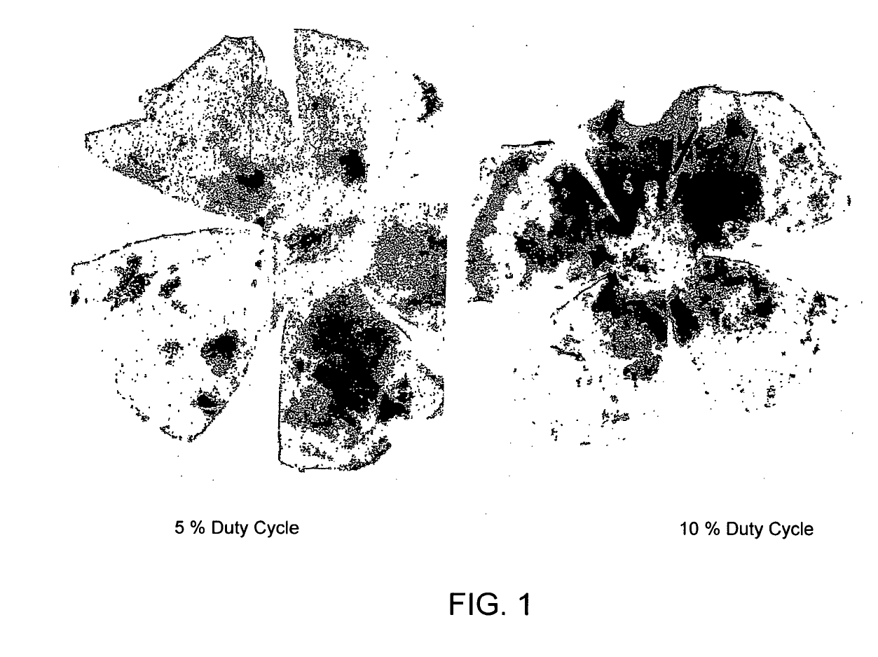

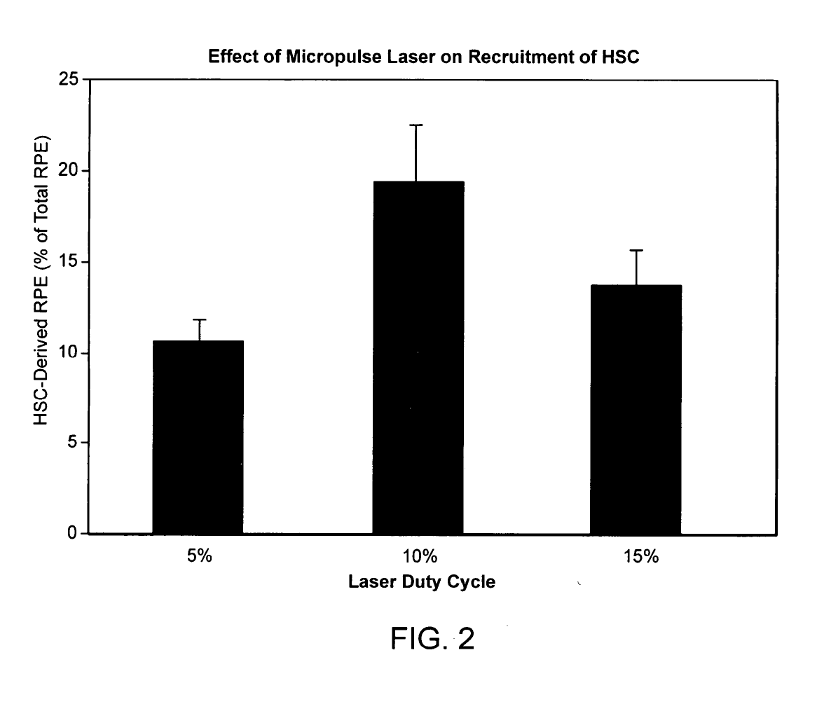

Eye cups were prepared and the neurosensory retina was removed. Eyes receiving subthreshold laser energy demonstrated robust recruitment of hematopoietic stem cells (HSC) to the retinal pigment epithelial layer in a diffuse pattern. These changes occurred within a 2 week period. Sub-threshold laser induced adult stem cells to migrate to and repair the retinal pigmented epithelium as shown in FIGS. 1 and 2. FIG. 1 demonstrates the striking duty-cycle dependent localization of gfp⁺ cells at the level of the RPE. The dark regions in FIG. 1 represent GFP⁺ cells that have incorporated into the RPE layer in areas that have received laser. Background fluorescence, as determined by the contralateral (unaffected) eyes was removed from this image. FIG. 2 is a graph quantitating human stem cell (HSC) incorporation into RPE.

### Example 2

### HSC's Recruited to the Retina Express a Retinal Specific Marker

By confocal immunofluorescence microscopy, it was also shown that GFP positive cells co-localized with RPE65—a protein specific for the RPE, suggesting that the recruited hematopoietic stem cells have acquired RPE characteristics. These eyes also demonstrated diffuse endothelial cell recruitment as well. Eyes receiving high energy laser with noticeable retinal opacification (150 mW, 0.1 sec) showed focal recruitment of GFP⁺ HSC to the scar region along with endothelial cells. Sub-threshold laser induced HSC to migrate to and incorporate into the RPE. The degree of incorporation correlates with laser duty cycle. 15% duty cycle resulted in the greatest degree of HSC incorporation.

### Example 3

### Subvisible Threshold Laser Increased Hsp70, Hsp90, and Induction of the Heat Shock Response Resulted in the Release of SDF-1 and VEGF

Ophthalmic lasers are an important tool for the treatment of various retinal disorders. In most instances, the effect has been attributed to visible changes in the retina (i.e., laser-induced photochemical burns). The diode 810 nanometer laser is believed to cause less damage to the neurosensory retina because the laser energy is absorbed by the RPE. FIG. 2 demonstrated the striking duty-cycle dependent localization of GFP⁺ cells at the level of the RPE. There is a maximal response at 10% duty-cycle. This was separately confirmed using adoptive transfer methods, a technique that closely resembles cellular therapy. Adoptive transfer involves the systemic administration of HSCs and results in the rapid homing of these cells to areas producing chemoattractants.

Micropulse lasering has been developed clinically to minimize photodestructive damage to the retina. The infrared (810 nm) laser used in micropulse mode is a relatively new modality for potential treatment of retinal disorders. Using repetitive, brief pulses of laser during a single exposure limits the amount of heat conduction and subsequent RPE damage. It has recently gained clinical acceptance for this reason. The heat shock response is considered to be a cytoprotective response based on the ability of the ensemble of heat shock proteins to limit protein misfolding.

To determine whether the use of micropulse lasering acted through thermal effects that induce the heat shock response and/or induction of cytokine and growth factors that attract HSCs to the eye, an infrared laser with variable duty cycle was used to examine the time course of mRNA expression of hsp70, hsp90 and crystallins in both the neurosensory retina and the posterior cup which contains the RPE and choroid complex. A peak increase in hsp70 was observed two hours post-laser in the neural retina and four hours post-laser in the posterior eye cup. mRNA for hsp90 dramatically peaked in both the neural retina and the posterior eye cup at two hours. Laser-induced expression of SDF-1 and its receptor CXCR-4 in the posterior eye cup was also observed. Examination at two hours post-laser suggested that a brief laser treatment effected the RPE cell's transcriptional machinery and reprogrammed the RPE cell to produce a series of factors that are capable of recruiting HSCs to the retina. By 4 hours post-laser, an increase in expression of SDF-1 and CXCR-4 was observed. Since SDF-1 and VEGF are known to be responsive to hypoxia, the effect of lasering the retina on HIF-1α mRNA levels was examined. mRNA for HIF-1α was reduced at 2 hours and increased at 4 hours in the posterior eye cup.

These studies support the autocrine and paracrine regulation of RPE by SDF-1 and VEGF. Without wishing to be bound by theory, it is likely that subvisible laser primes the extracellular environment of the RPE—photoreceptor layers and creates a receptive environment for recruiting HSCs. Growing evidence indicates that extracellular hsp70 is a neuroprotective agent. Without wishing to be bound by theory, hsp70 may be acting as a neuroprotective a factor in the retina by facilitating the recruitment of HSCs to the retina to provide for the repair of the RPE. The heat shock proteins likely function in the recruitment of HSC to the retina. This may be accomplished in vivo by the recruitment of HSCs following local production of chemoattractant proteins.

### Example 4

### Hsp70 mRNA Levels Increased Following Heat Shock of Primary RPE Cultures

To determine if the RPE could be a source of the observed in vivo cytokine response, cultures of human primary RPE and ARPE19 (an immortalized RPE cell) were heat shocked. At two hours post heat-shock, suppression of the mRNAs of the heat shock proteins, HIF-1α as well as SDF-1 and VEGF was observed. In RPE cultures, a dramatic forty-fold increase in hsp90 mRNA levels was observed. The in vitro hsp90 results paralleled results in vivo. Strikingly, a fifty-fold increase in hsp70 mRNA levels was observed in the primary RPE cultures, which indicated that resident RPE cells released this putative neuroprotective agent. Based on these in vivo data, and without wishing to be bound by theory, it is likely that RPE cells are the source of chemotactic factors that facilitated HSC recruitment to the retina. This does not preclude the possibility that other cell types participated in the recruitment response.

In sum, these results indicated for the first time that HSCs can be locally recruited to the retina, including the RPE layer by either laser or pharmacological induction. This was achieved with SVL induction of the heat shock response. The laser-induced heat shock response was temporally associated with the release of HIF-1α and then the HSC chemoattractants SDF-1 and VEGF. The present results produced no clinically visible laser burn or scar. This lack of damage distinguishes the present methods from methods that induce visible retinodestructive lasering.

### Example 5

### Chemically Induced Heat Shock Recruits HSC to the RPE

These observations were extended by chemically inducing the heat shock response in primary human RPE cells. Four hours post-laser exposure, there was an exuberant increase in hsp70 levels, and a moderate increase in hsp90, hsp32 and crystallin mRNA levels. SDF-1 expression was also observed. The time course for this expression mirrored that seen during classic heat shock induction.

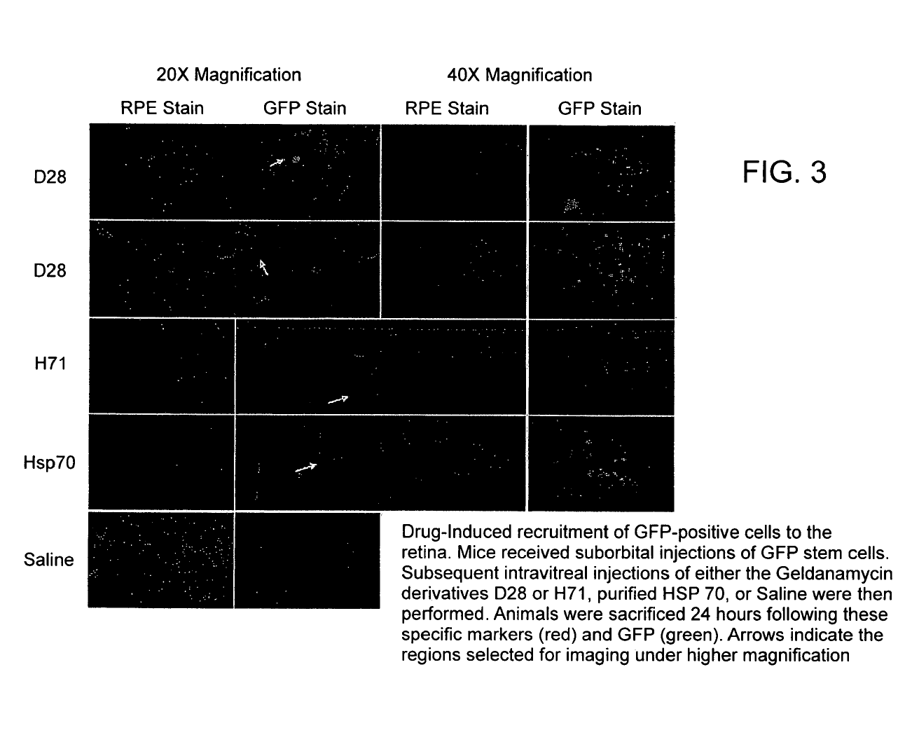

In addition, chemically induced heat shock recruited HSC to the RPE as shown in FIG. 3. The pharmacological induction with small molecule inducers of the heat shock response was induced by the intravitreal injection of geldanamycin analogs or by separately exposing RPE cells resulted in the identical induction of SDF-1 and VEGF. These experiments provide conclusive evidence that heat shock induction, by either laser or pharmacological induction, directly results in the production HIF-1α and the critical HSC chemokines, SDF-1 and VEGF. Further, they suggest pharmacological manipulation effectively leads to HSC recruitment to the RPE layer and differentiation.

### Example 6

### Chemically Induced Heat Shock Induces the Expression of a Powerful Stem Cell Attractant

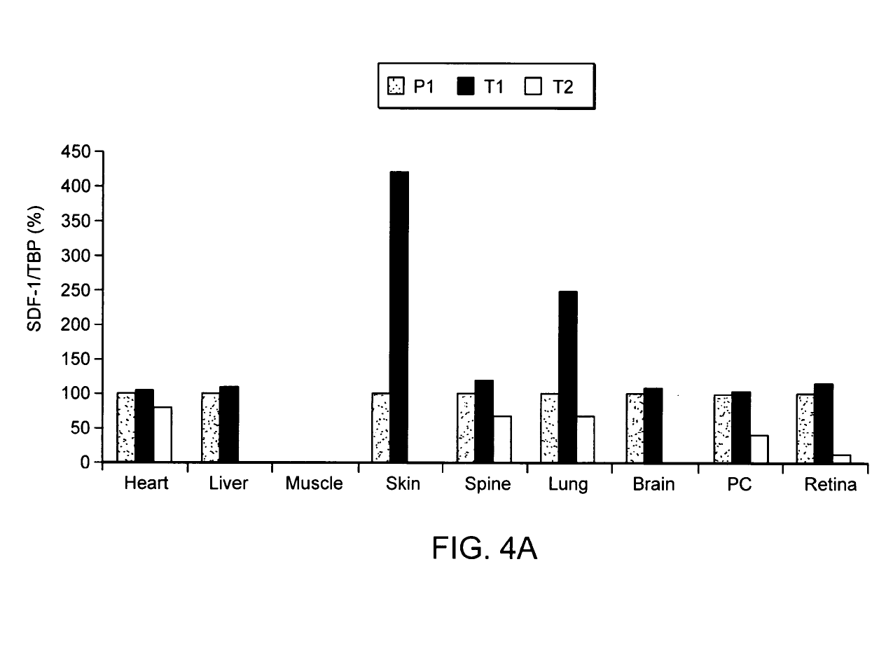

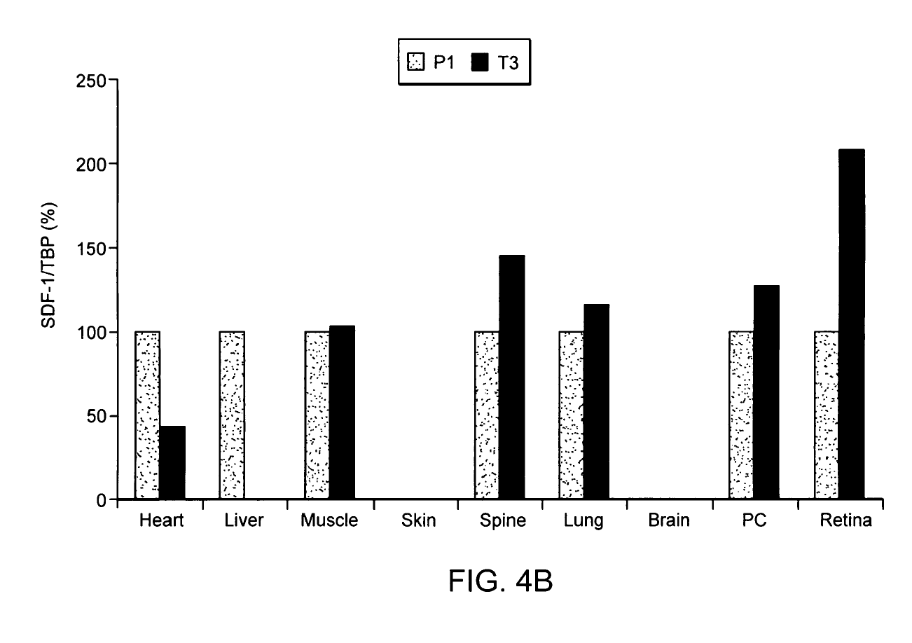

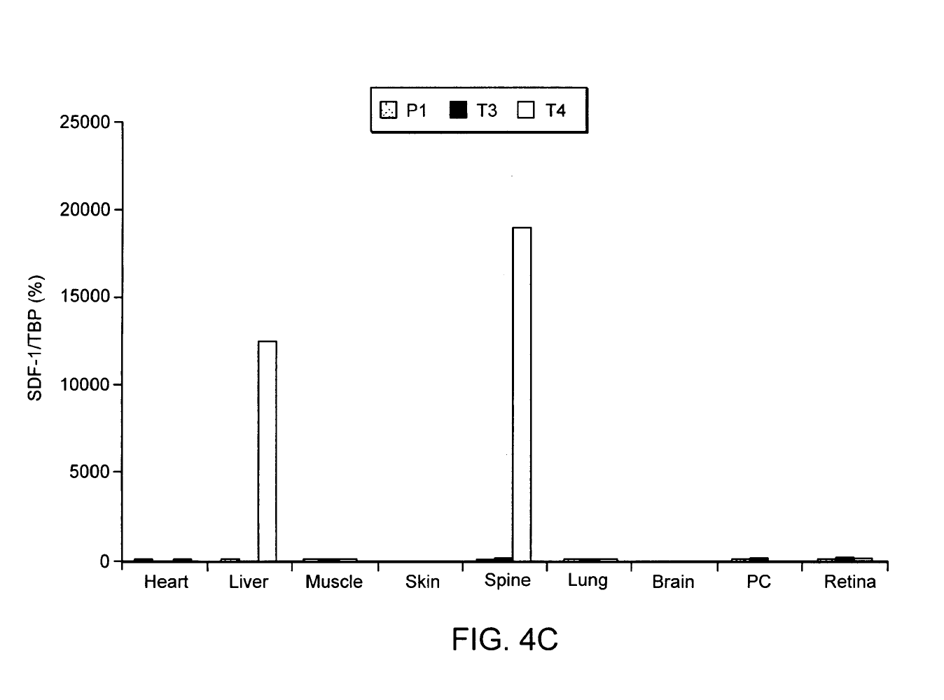

Mice were injected with celastrol a chemical inducer of the heat shock response intraperitoneally. Mice organs were harvested at days 1, 2, 3, and 4 post-injection. RT-PCR was performed of the RNA isolated from tissues for SDF-1, which is the most potent chemoattractant for endogenous stem cells known. The bar graphs compare placebo to the treated mice in the various tissues at various times after injection. FIG. 4A shows that SDF-1 is strongly induced by chemical heat shock within twenty-four hours in skin and lung. Increases in SDF-1 are also seen at twenty-four hours in a variety of other tissues (FIG. 4A). FIG. 4B shows that increases in SDF-1 expression are observed three days after celastrol injection in muscle, spine, lung, posterior cup, and retina. FIG. 4C shows that a large increase in SDF-1 expression is observed at three and four days after celastrol injection in liver and spine.

The present invention provides laser and pharmacological methods for the treatment of diseases, disorders, or injuries characterized by a deficiency in cell number. The present methods further provide hematopoietic stem cell therapy for diseases characterized by a deficiency in cell number. The present studies suggest that such diseases may be treated by enhancing a tissue repair function using a combination of laser and pharmacological approaches. In particular, the invention provides methods for priming patients with an agent that mobilizes HSCs, such as GM-CSF, followed by sub-visible laser or injection of compounds that can induce the heat shock response and initiate cellular repair of a tissue or organ in need thereof.

The results described above were obtained using the following methods and materials.

### Electroretinography (ERG)

Retinal function of treated and untreated eyes is evaluated by ERG (a non-invasive technique used to determine photoreceptor function) on a periodic (e.g., monthly) basis to determine the effect of laser or pharmacological agent therapy. Electroretinography is a non-invasive technique in which the corneal electrical response to light is measured in anesthetized animals. Mice are anesthetized with intraperitoneal injections of a mix of 80-100 mg/kg ketamine and 5-10 mg/kg xylazine for anesthesia (Phoenix Pharmaceuticals, St. Joseph, Mo.). The mouse corneas are anesthetized with a drop of 0.5% proparacaine HCl (Akorn, Buffalo Grove, Ill.), and dilated with a drop of 2.5% phenylephrine HCl (Akorn). Measurement electrodes tipped with gold wire loops are placed upon both corneas with a drop of 2.5% hypromellose (Akorn) to maintain electrode contact and corneal hydration. A reference electrode is placed subcutaneously in the center of the lower scalp of the mouse, and a ground electrode is placed subcutaneously in the hind leg. The mouse rested on a homemade sliding platform that keeps the animal at a constant temperature of 37° C. The animal is positioned so that its entire head rested inside of the Ganzfeld (full-field) illumination dome of a UTAS-E 2000 Visual Electrodiagnostic System (LKC Technologies, Inc., Gaithersburg, Md.). Full-field scotopic ERGs are measured by 10 msec flashes at an intensity of 0.9 and 1.9 log cd m−2 at 1 minute intervals.

Responses are amplified at a gain of 4,000, filtered between 0.3 to 500 Hz and digitized at a rate of 2,000 Hz on two channels. Five responses are averaged at each intensity. The wave traces analyzed using UTAS-E 2000 software package (LKC Technologies, Inc.). A-waves are measured from the baseline to the peak in the cornea-negative direction; b-waves are measured from the cornea-negative peak to the major cornea-positive peak.

The animals receive triple antibiotic ointment (Vetropolycin) in their eyes to maintain moisture following the procedure, and are allowed to regain consciousness on a 37 degree warming tray before they are returned to the vivarium. Animals receiving Ketamine/Xylazine anesthesia will also receive 0.01-0.02 ml/g of body weight of warm LRS SQ.

### Funduscopy

Retinal examination of treated and untreated eyes is evaluated by funduscopic examination. Funduscopy is a non-invasive technique in which retinal photographs are taken of anesthetized animals. Mice are anesthetized, and their corneas are anesthetized and dilated as described above for ERG analysis. Fundus photography is performed with a specialized camera and lens, a Kowa Genesis hand held fundus camera (Kowa Company, Ltd., Tokyo, Japan) focused through a Volk Super 66 Stereo Fundus Lens (Keeler, Berkshire, England). Two pictures of each eye are generally taken to ensure a properly focused image. The animals receive triple antibiotic ointment (Vetropolycin) in their eyes to maintain moisture following the procedure, and are allowed to regain consciousness on a 37° C. warming tray before they are returned to the vivarium. Animals receiving Ketamine/Xylazine anesthesia will also receive 0.01-0.02 ml/g of body weight of warm LRS SQ.

### Laser Treatment

The pre-surgical preparation of the animals involves making sure that they are physically active and able to undergo anesthesia. The mice need to be without any evidence of ocular discharge or evidence of a cataract so as to make it feasible to visualize the retina to perform laser burn treatment. Prior to the laser treatment the animals will be anesthetized with intraperitoneal injection of a mix of 80-100 mg/kg ketamine and 5-10 mg/kg xylazine. No corneal edema or cataract formation is attributed to the use of these anesthetics. The level of anesthesia is monitored by a footpad pinch and breathing rate. A lack of the reflex-response to the footpad pinch indicates that the animal is properly anesthetized. No ocular ointment is applied before or during the laser treatment because this would prevent effective laser treatment. Some antibiotic ointment is applied to protect the untreated eye. If no response is demonstrated after footpad pinch then the laser treatment will proceed.

Pain, distress or discomfort is suggested by movement of the animal during the time required for laser treatment. If the animal shows increased movement just prior to or during laser surgery then anesthesia is supplemented by exposing the animal to isoflurane for 10 seconds. Approximately 1 mL isoflurane is soaked onto a crumpled Kimwipe placed in the bottom of a 50 mL plastic centrifuge tube and the tube is capped. If necessary the open end of this tube can be held briefly near the animal's nose. This procedure is performed in a fume hood. After this the animals' breathing rate will continue to be monitored and the animal's pain response will be monitored by footpad pinch. If no movement is demonstrated after footpad pinch, then the laser surgery will proceed. The laser treatment takes approximately 30 seconds per mouse. An intraperitoneal injection of yohimbine (2 mg/kg body weight) is used to reverse the effect of the ketamine/xylaxine. This will reduce the amount of time that the eyes are at risk due to the loss of the blink reflex under anesthesia. No abnormal behavior is expected following surgery.

Pain, distress and discomfort can occur after laser treatment. The literature indicates that human recovery from laser treatment is helped by application of ketorolac to the cornea at the end of the procedure (Kosrirukvongs et al, Topical ketorolac tromethamine in the reduction of adverse effects of laser in situ keratomileusist, J. Med Assoc That, 2001; 84:804-810 and Price, et al, Pain reduction after laser in situ keratomileusis with ketorolac tromethamine ophthalmic solution 0.5%: a randomized, double-masked, placeb0-controlled trail, J. Refeact Surg, 2002:18:140-144). Therefore, after laser treatment drops of a solution of ketorolac (0.5% OP) will be applied to the eyes of the mice for 48 hours following treatment, and longer if needed. The commercial name of this solution is Acular PF Solution. This duration of treatment with Acular PF has no adverse effects on the mice and has no effect on neovascularization as evidenced by analysis of the vehicle injected animals. The animals will be maintained in their cages and held at room temperature (18-26° C.), or on a 37° C. warming tray and visually monitored continuously until they show signs of recovery from anesthesia. Full recovery from anesthesia occurs only when the animal is fully alert and ambulatory in the cage.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

This application contains subject matter that may be related to subject matter described in U.S. Provisional Application Nos.: 60/703,068, which was filed on Jul. 27, 2005, and 60/729,182, which was filed on Oct. 21, 2005; and the International application filed on Jul. 27, 2006, entitled “Use of Heat Shock to Treat Ocular Disease,” (application number not yet assigned), the entire contents of each of these applications is hereby incorporated by reference.

All patents and publications mentioned in this specification are herein incorporated by reference to the same extent as if each independent patent and publication was specifically and individually indicated to be incorporated by reference.

1. A method for ameliorating a disease characterized by a deficiency in cell number or recruiting a stem cell to a tissue in a subject, the method comprising
(a) inducing heat shock in at least one cell of a tissue having a deficiency in cell number using a small compound; and
(b) recruiting a stem cell to the tissue, thereby ameliorating the disease.

2. (canceled)

3. The method of claim 1, wherein the small compound is selected from the group consisting of geldanamycin, celastrol, 17-allylamino-17-demethoxygeldanamycin, ECl 02, radicicol, geranylgeranyl acetone, paeoniflorin, PU-DZ8, and H-71.

4. The method of claim 3, wherein the method increases the expression or activity of a heat shock protein selected from the group consisting of Hsp100, Hsp90, Hsp70, Hsp60, and Hsp40.

5. (canceled)

6. The method of claim 1, wherein the stem cell is a hematopoietic stem cell.

7. (canceled)

8. The method of claim 1, wherein the tissue is selected from the group consisting of bladder, brain, nervous tissue, glia, esophagus, fallopian tube, heart, pancreas, intestines, gall bladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage.

9-18. (canceled)

19. The method of claim 6, wherein the method alters the expression or activity of a protein selected from the group consisting of SDF-I, VEGF, HIF-Iα, crystallin, hypoxia-inducible factor 1-alpha (HIF-Iα), and CXCR-4.

20-34. (canceled)

35. The method of claim 1, wherein the disease is selected from the group consisting of ischemic injury, myocardial infarction, muscle ischemia, a neural, glial, or muscle degenerative disorder, muscular atrophy or dystrophy, heart disease, congenital heart failure, hepatitis, cirrhosis of the liver, an autoimmune disorder, diabetes, cancer, a congenital defect that results in the absence of a tissue or organ, angina pectoris, myocardial infarction, ischemic limb, accidental tissue defect, fracture or wound.

36. A method of ameliorating a disease or disorder in a subject in need thereof, the method comprising
(a) administering to the subject an agent that mobilizes a bone marrow derived stem cell in the subject;
(b) inducing heat shock in a tissue having a deficiency in cell number using a small compound; and
(c) recruiting the stem cell to the tissue, thereby ameliorating the disease or disorder.

37. The method of claim 36, wherein the agent is granulocyte macrophage colony stimulating factor or stem cell factor.

38-39. (canceled)

40. The method of claim 36, wherein the small compound is selected from the group consisting of geldanamycin, celastrol, 1T-allylamino-1V-demethoxygeldanamycin, ECl 02, radicicol, geranylgeranylacetone, paeoniflorin, PU-DZ8, and H-71.

41. The method of claim 36, wherein the disease is selected from the group consisting of ischemic injury, myocardial infarction, muscle ischemia, a neural, glial, or muscle degenerative disorder, muscular atrophy or dystrophy, heart disease, congenital heart failure, hepatitis, cirrhosis of the liver, an autoimmune disorder, diabetes, cancer, a congenital defect that results in the absence of a tissue or organ, angina pectoris, myocardial infarction, ischemic limb, accidental tissue defect, fracture or wound.

42-53. (canceled)

54. The method of claim 1 or 36, wherein the cell is not an ocular cell.

55. (canceled)

56. The method of claim 1 or 36, further comprising locally or systemically administering an isolated stem cell to enhance tissue repair or regeneration.

57. A pharmaceutical composition for recruiting stem cells to a tissue, the composition comprising an effective amount of a small compound selected from the group consisting of geldanamycin, celastrol, 17-allylamino-17-demethoxygeldanamycin, ECl 02, radicicol, geranylgeranylacetone, paeoniflorin, PU-DZ8, and H-71 or an expression vector comprising a polynucleotide encoding a heat shock polypeptide in a pharmaceutically acceptable excipient.

58-60. (canceled)

61. A kit comprising an effective amount of an agent that induces a heat shock response in a tissue, and instructions for using the kit to increase stem cell recruitment in the tissue.

62. (canceled)

63. The kit of claim 61, wherein the agent is a polypeptide selected from the group consisting of Hsp 100, Hsp90, Hsp70, HspóO, 5 and Hsp40, or a polynucleotide encoding said polypeptide, or a small compound that increases the expression or activity of said polypeptide.

64-67. (canceled)
